(19) United States
(12) Patent Application Publication
(10) Pub. No.: US 2007/0179611 A1
(43) Pub. Date: Aug. 2, 2007

(54) METHODS AND DEVICES FOR REPLACEMENT OF INTERVERTEBRAL DISCS

(76) Inventors: Gene P. DiPoto, Upton, MA (US); Alan E. Shluzas, West Roxbury, MA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/615,762

(22) Filed: Dec. 22, 2006

Related U.S. Application Data
(60) Provisional application No. 60/753,244, filed on Dec. 22, 2005.

(51) Int. Cl. A61F 2/44

(52) U.S. Cl. 623/17.11

(57) ABSTRACT

An artificial vertebral disc for replacing an intervertebral disc of a patient is provided. The artificial disc can comprise a first plate, a second plate, and an elongate member extending between the first plate and the second plate. The elongate member is preferably flexible to allow angulation between the plates. In one embodiment, a side support extends along the exterior of the disc to substantially enclose the disc. In another embodiment, the disc has an open configuration and a stopping member to limit the angulation of one plate relative to another plate. The disc can include an insert configured to be inserted in a space defined by the stopping member and one of the plates to prevent movement of the plates. Methods for replacing an intervertebral disc in an interbody space of a spine using the disclosed artificial discs are also provided.

### CROSS-REFERENCE TO RELATED APPLICATIONS

This application claim priority under 35 U.S.C. § 119(e) to U.S. Provisional Application No. 60/753,244, filed on Dec. 22, 2005, the entirety of which is hereby incorporated by reference.

### BACKGROUND OF THE INVENTION

1. Field of the Invention

The present invention relates generally to replacing an intervertebral disc of a patient with a prosthetic device. More specifically, certain embodiments of the invention relate to artificial vertebral discs having an elongate member that is flexible to allow angulation between plates of the disc.

2. Description of the Related Art

Spinal discs can become damaged by injury or can degenerate over time. As a result, the natural spacing between vertebral bodies is often impaired and adjacent vertebrae typically contact each other or contact nerves running through the spine thereby causing pain and discomfort. Artificial vertebral discs have been created to replace damaged spinal discs and to provide a support medium between adjacent vertebral bodies.

### SUMMARY OF THE INVENTION

In one embodiment, an artificial disc for replacing an intervertebral disc of a patient comprises a first plate configured to contact a first vertebra and a second plate configured to contact a second vertebra. An elongate member comprising a center post extends between the plates and is flexible to allow angulation between the plates.

In another embodiment, an artificial disc for replacing an intervertebral disc of a patient comprises a first plate configured to contact a first vertebra and a second plate configured to contact a second vertebra. An elongate member extends between the plates and allows the second plate to move relative to the first plate. A side support extends around the exterior of the artificial disc to substantially enclose the disc. At least a portion of the side support is compressible.

In another embodiment, an artificial disc for replacing an intervertebral disc comprises a first plate configured to contact a first vertebra of the spinal column and a second plate configured to contact a second vertebra. An elongate member connects the plates and allows one plate to move relative to the other plate. A stopping member is attached to one of the plates and is spaced apart from the other plate. The stopping member limits the angulation of the other plate of the artificial disc.

An additional embodiment involves a method of replacing an intervertebral disc in an interbody space of a spine of a patient. The method comprises inserting an artificial vertebral disc in the interbody space between a first vertebra and a second vertebra, wherein the artificial disc comprises a first plate that contacts the first vertebra and a second plate that contacts the second vertebra and an elongate member comprising a center post extending between the first plate and the second plate, wherein the elongate member is flexible to allow angulation between the plates.

### BRIEF DESCRIPTION OF THE DRAWINGS

These and other features, embodiments, and advantages of the present invention will now be described in connection with preferred embodiments of the invention, in reference to the accompanying drawings. The illustrated embodiments, however, are merely examples and are not intended to limit the invention. The drawings include seventeen figures, which are briefly described as follows:

FIG. 1 is a perspective view of an artificial vertebral disc having an enclosed configuration used for total intervertebral disc replacement in accordance with an embodiment of the present invention.

FIG. 2 is top plan view of the artificial vertebral disc as shown in FIG. 1 showing an elongate member centrally located within the disc and a side support extending around the exterior of the disc.

FIG. 3 is a sectional view of the artificial vertebral disc of FIG. 2 taken along line 3-3 of FIG. 2.

FIG. 3A is a sectional view of the artificial vertebral disc taken along line 3-3 of FIG. 2 showing a force applied to one portion of the disc.

FIG. 3B is a sectional view of the artificial vertebral disc taken along line 3-3 of FIG. 2 showing a force applied to another portion of the disc.

FIG. 4 is a top plan view of an embodiment of the artificial vertebral disc having an open configuration showing an elongate member centrally located within the disc and stopping members on opposite sides of the elongate member.

FIG. 5 is a sectional view of the artificial vertebral disc taken along line 5-5 of FIG. 4.

FIG. 5A is a sectional view of the artificial vertebral disc taken along line 5-5 of FIG. 4 showing a force applied to one portion of the disc.

FIG. 5B is a front cross-sectional view of the artificial vertebral disc taken along line 5-5 of FIG. 4 showing a force applied to another portion of the disc.

FIG. 6 is a top plan view of an embodiment of the artificial vertebral disc with an open configuration having an insert used for total intervertebral disc replacement in bone fusion applications in accordance with an embodiment of the present invention. In this figure, the insert is shown in a disengaged position.

FIG. 6A is a top plan view of the artificial vertebral disc of FIG. 6 showing the insert in an engaged position.

FIG. 7 is a sectional view of the artificial vertebral disc of FIG. 6A taken along line 7-7 of FIG. 6A.

FIG. 8 is a top plan view of an embodiment of the artificial vertebral disc with an open configuration having a series of stopping members to limit angulation of the plates.

FIG. 9 is a top plan view of an embodiment of the artificial vertebral disc with an open configuration having a wall extending around the exterior of the disc to limit angulation of the plates.

FIG. 10 is a perspective view of one embodiment of an access device.

FIG. 11 is a schematic view of one surface of a vertebra that defines one end of an interbody space and one embodiment of an access device configured to provide access to the interbody space.

FIG. 11A is a cross-section view of a proximal portion of one embodiment of an access device of FIG. 11.

FIG. 11B is a cross-section view of a proximal portion of another embodiment of the access device of FIG. 11.

FIG. 12 is a schematic lateral view of a portion of a spine with the access device of FIG. 11B applied thereto to provide access to an interbody space.

FIG. 13 is a schematic view similar to that of FIG. 11 illustrating one method of inserting a spinal implant into an interbody space through an access device.

FIG. 14 is a schematic view similar to that of FIG. 12 showing a spinal implant configured to preserve or restore motion inserted into an interbody space.

FIG. 15 is a schematic posterior view of a portion of a spine with an access device applied thereto to insert a guide to an interbody space.

FIG. 16 is a perspective view of one embodiment of a guide attached to a vertebra, facilitating access to an interbody space.

FIG. 17A is a view of one method of preparing an interbody space for the insertion of a spinal implant into an interbody space using a guide.

FIG. 17B is a cross-sectional view of a path shown in FIG. 17A.

FIG. 17C is a lateral view of a portion of a spine with an access device, guide and mill applied thereto for preparing an interbody space for a spinal implant.

Throughout the figures, the same reference numerals and characters, unless otherwise stated, are used to denote like features, elements, components or portions of the illustrated embodiments. Moreover, while the subject invention will now be described in detail with reference to the figures, it is done so in connection with the illustrative embodiments. It is intended that changes and modifications can be made to the described embodiments without departing from the true scope and spirit of the subject invention as defined by the appended claims.

### DETAILED DESCRIPTION OF THE PREFERRED EMBODIMENTS

As should be understood in view of the following detailed description, this application is primarily directed to apparatuses and methods for providing access to and for treating the spine of a patient. The apparatuses described below provide access to surgical locations at or near the spine and provide a variety of artificial vertebral discs for replacing a damaged intervertebral disc of a patient. In particular, various embodiments described hereinbelow include artificial vertebral discs that are particularly well adapted for replicating the natural characteristics of an intervertebral disc. In some embodiments, intervertebral discs are provided that have a substantially enclosed configuration. In other embodiments, intervertebral discs are provided that have an open configuration. The apparatuses and methods described enable a surgeon to perform a wide variety of methods of treatment for replacing an intervertebral disc of a patient as described herein.

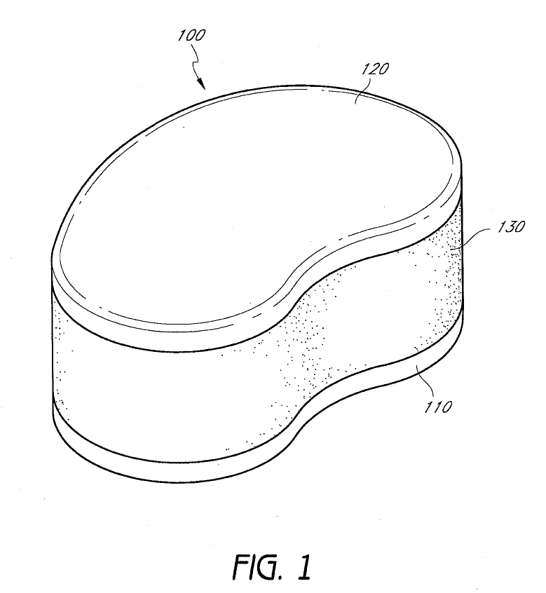

As shown in FIG. 1, an artificial vertebral disc 100 having a substantially closed configuration is provided. The artificial vertebral disc 100 comprises a first plate 110 and a second plate 120. The disc 100 also has a side support 130 connected to and extending between the first plate 110 and second plate 120. The side support 130 preferably extends around the exterior of the artificial disc 100 and substantially encloses the disc 100. The side support 130 can provide support for the plates 110, 120. In one embodiment, the side support 130 comprises a side wall. The first plate 110, in one embodiment, comprises a lower plate 110 and the second plate 120 comprises an upper plate 120 disposed above the lower plate 110.

When the artificial disc 100 is inserted in a disc space, the upper plate 120 provides support for an upper vertebral body of the spinal column (which is located adjacent the disc space and above the disc 100) and the lower plate 110 rests on a lower vertebral body (which is located adjacent the disc space and below the disc 100) such that the artificial disc 100 can be implanted at least partially between adjacent vertebral bodies so as to replace damaged discs in the spine and maintain the natural spacing between adjacent vertebrae. In one embodiment, the first plate 110 and the second plate 120 are composed of a metal alloy, preferably a titanium alloy.

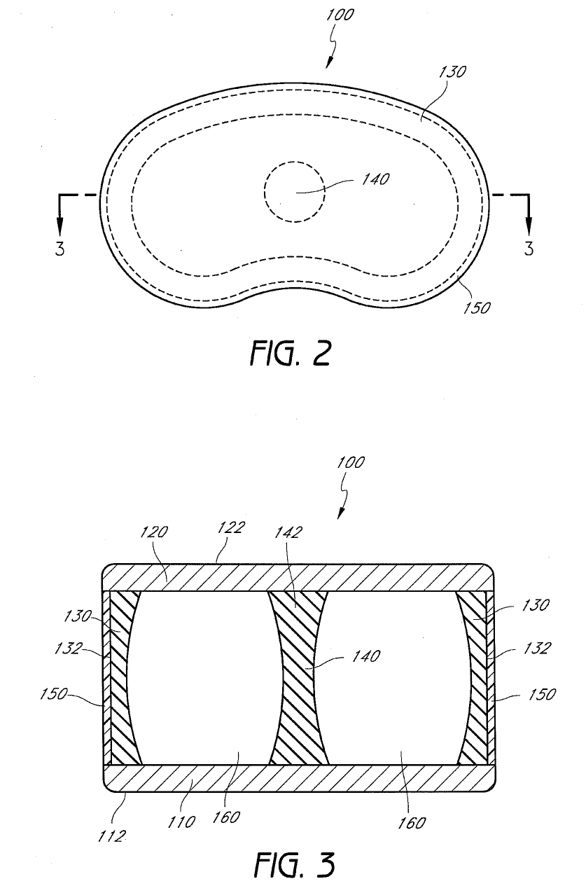

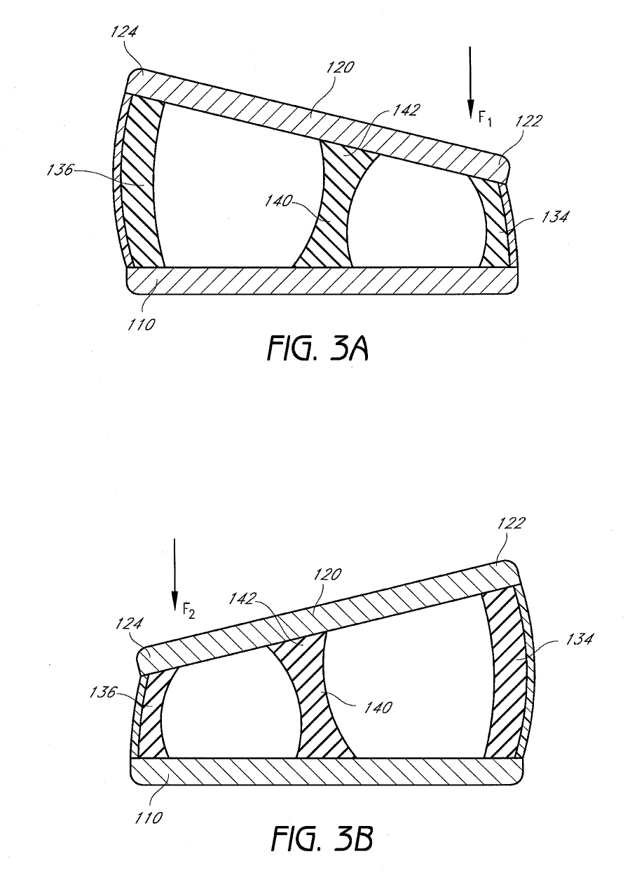

With reference to FIGS. 2 and 3, the disc 100 has a side support 130 such that the first plate 110, second plate 120, and the side support 130 define an enclosed region. As seen in FIG. 2, the side support 130 preferably extends along substantially the entire exterior of the disc 100 such that the disc 100 comprises an enclosed space defined by the first plate 110, the second plate 120, and the side support 130. The side support 130, in one embodiment, can provide support for the upper plate 120. At least a portion of the side support 130 can be compressible thus permitting the disc 100 to conform to the natural spacing of adjacent vertebral bodies.

An elongate member 140 is centrally disposed between the first plate 110 and the second plate 120. The elongate member 140 extends between the first plate 110 and second plate 120, and it is preferably connected to the plates 110, 120. The elongate member 140 allows the second or upper plate 120 to move relative to the first or lower plate 110. In one embodiment, the elongate member 140 preferably is flexible, but not compressible, such that the elongate member 140 acts as a center column providing support to an upper vertebral body resting on the second or upper plate 120. In one embodiment, the elongate member 140 comprises a center post generally disposed in a central portion of the disc 100. The center post 140, in one embodiment, can have a substantially continuous outer surface extending between the plates 110, 120 (which can be seen in FIG. 3).

The elongate member or center post 140 can be composed of a shape memory alloy such as nickel-titanium. The flexible nature of the elongate member 140 permits the second plate 120 to at least partially pivot about an upper portion 142 of the elongate member 140 as shown below in FIGS. 3A and 3B. Thus, the flexible elongate member 140 allows angulation between the plates.

In another embodiment, the elongate member 140 comprises a spring member such that the member 140 is compressible allowing the artificial disc to compress upon application of a compressive force. Similarly, in yet another embodiment, the elongate member 140 comprises a dashpot also enabling compression of the member 110.

The artificial disc 100 also includes a cavity 160 defined by the first plate 110, the elongate member 140, the second plate 120, and the side support 130. The cavity 160 is preferably composed of a rubberized material such as Silicon or hydrogel in order to replicate the natural characteristics of intervertebral discs. As a result, the cavity 160 allows for compression of the disc 100 and provides sufficient support for the second or upper plate 120 upon application of force by an upper vertebral body.

The side support 130 may act as an impermeable barrier so as to prevent rubberized material of the cavity 160, as described above, from leaking from the disc 100. In one embodiment, the side support 130 comprises substantially the same material as the rubberized material of the cavity 160. In another embodiment, the side support 130 comprises an alternative material preferably having impermeable characteristics as well.

A sheath 150 covers an outer surface 132 of the side support 130. The sheath 150 preferably extends around the exterior of the prosthetic disc 100 along the outer surface 132. Advantageously, the sheath 150 provides an additional barrier so as to prevent the rubberized material of the cavity 160 from leaking from the prosthetic disc 100 in a situation where rubberized material penetrates the side support 130. In one embodiment, the sheath 150 comprises Goretex.

Although not shown in FIGS. 1 through 3, the outer surfaces 112, 122 of the first plate 110 and the second plate 120 can include means for securing the first and second plates 110, 120 to adjacent vertebral bodies. Such means can include, among other things, rough surfaces adapted to engage vertebrae or one or more spikes.

As shown in FIGS. 3A and 3B, the elongate member 140 is preferably flexible such the second plate 120 can pivot about the upper portion 142 of the elongate member 140. In order to permit the second plate 120 to angulate with respect to the first plate 110, in one embodiment, the side support 130 is compressible. That is, the side support 130 can compress upon application of a given force such that the second plate 120 lies at an angle with respect to the first plate 110. Therefore, the height of the side support 130 can vary along the exterior of the disc 100 depending on the particular force that is applied to the disc 100.

For example, as shown in FIG. 3A, a longitudinal force F₁ can be applied to a first portion 122 of the second plate 120. The force F₁ will press the second plate 120 downward toward the first plate 110. In one embodiment, a first portion 134 of the side support 130 can be compressed in response to the applied longitudinal force F₁. As a result, the first portion 134 of the side support 130 will have a shorter height than a corresponding second portion 136 of the side support 130. The second portion 136 of the side support 130 can extend upward in order to accommodate compression of the first portion 134. In FIG. 3A, the elongate member 140 flexes and the side support 130 compresses and extends to accommodate a lordotic curvature, or forward curvature, of the spine.

With reference to FIG. 3B, a longitudinal force F₂ can similarly be applied to a second portion 124 of the second plate 120. In this embodiment, the force F₂ will press the second plate 120 downward toward the first plate 110 and a second portion 136 of the side support 130 is compressed in response to the applied longitudinal force F₁. Here, the elongate member 140 flexes and a second portion 136 of the side support 130 compresses, while the first portion 134 extends upward, in order to accommodate a kyphotic curvature, or rearward curvature, of the spine.

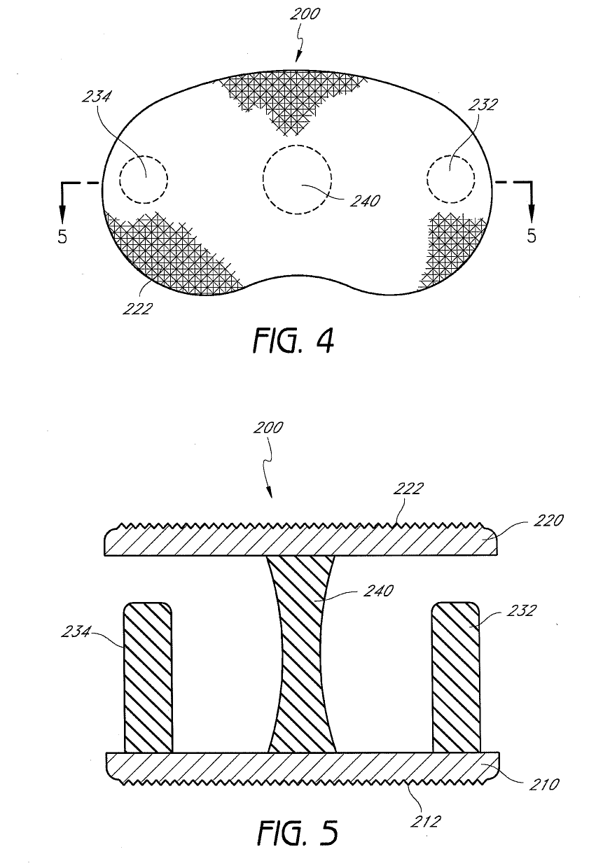

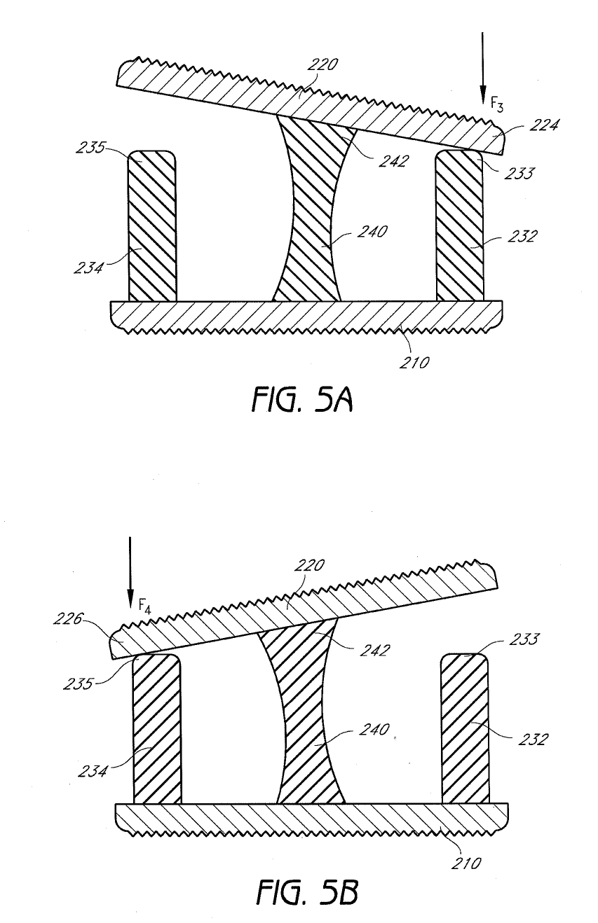

With reference to FIGS. 4 and 5, an artificial disc 200 with a non-enclosed body, or open body, is provided. The structure of the artificial disc 200 with an open body is similar to that of the artificial disc 100 with an elongate member having an enclosed configuration as shown in FIGS. 1 through 3. However, one difference between the two prosthetic discs is that the artificial disc 200 with a non-enclosed body is an open design such that its components preferably do not define an enclosed region within the disc 200.

The artificial disc 200 comprises a first plate 210, a second plate 220, and an elongate member 240. The first plate 210, or lower plate, has an outer surface 212 that is preferably rough so as to provide a means to secure the artificial disc 200 to a vertebral body. Likewise, the second plate 220, or lower plate, has an outer surface 222 that is preferably rough so as to provide a means to secure the artificial disc 200 to a lower vertebral body. In one embodiment, the means for securing the first and second plates 210, 220 to first and second vertebra comprises at least one spike.

The elongate member 240, in one embodiment, supports the second plate 220 and attaches the second plate 220 to the first or lower plate 210. The elongate member 240 generally extends between the two plates 210, 220. Similar to the elongate member 140 described above with reference to FIGS. 1 through 3, the elongate member 240 is preferably flexible so as to allow the second plate 220 to pivot about an upper portion 242 of the elongate member 240, and to allow angulation between the plates 210, 220. The elongate member 240 can comprise a nickel-titanium alloy in order to provide such desired flexibility. In other embodiments, the elongate member 240 can be compressible.

The artificial vertebral disc 200 can also include a stopping member to limit the angulation of the second plate 220 relative to the first plate 210. The stopping member can be attached to an inner surface of the first plate 210. The stopping member extends toward the second plate 220, but preferably does not come into contact with the second plate 220 when the disc 200 is in a resting position (i.e., the disc is not subject to any external forces). Thus, the stopping member is at least partially shorter than the elongate member 240 (which connects the first plate 210 and the second plate 220). The stopping member provides a barrier means, or stop, to prevent the second plate 220 from pivoting too far in one direction with respect to the first plate 210. In other embodiments, the stopping member may be attached to the second or upper plate 220 rather than the first or lower plate 210. The stopping member preferably limits angulation between the plates 210, 220.

In the illustrated embodiment, as shown in FIG. 5, a first stopping member 232 and a second stopping member 234 are provided. The stopping members 232, 234 can be disposed on opposite sides of the disc 200. For example, the first stopping member 232 can be disposed on a first side of the disc 200, while the second stopping member 234 can be positioned on the second side of the disc 200. In one embodiment of the artificial disc 200, the stopping members 232, 234 are similar in configuration to that of a cylindrical post.

FIGS. 5A and 5B show the disc 200 subject to applied forces F₃, F₄, respectively. In FIG. 5A, a compressive force F₃ is applied to a first side 224 of the upper plate 220. The force F₃ causes the second plate 220 to pivot about an upper portion 242 of the elongate member 240 and the first side 224 of the second plate 220 to displace downward. The first stopping member 232 prevents the first side 224 of the second plate 220 from displacing beyond a desired distance. Namely, at some point, the first side 224 will contact an upper surface 233 of the first stopping member 232. The upper surface 233 of the first stopping member 232 thus provides support for the second plate 220 upon application of a compressive force F₃. In this embodiment, the elongate member 240 flexes so that the disc 200 can accommodate a lordotic curvature of the spine.

Similarly, as illustrated in FIG. 5B, a compressive force F₄ can be applied to a second side 226 of the upper plate 220. The second stopping member 234 preferably prevents the second side 226 from displacing too far and supports the second plate 220 in this situation. An upper surface 235 of the second stopping member 234 provides support for the second plate 220 upon application of a compressive force F₄. In this embodiment, the elongate member 240 flexes to accommodate a kyphotic curvature of the spine.

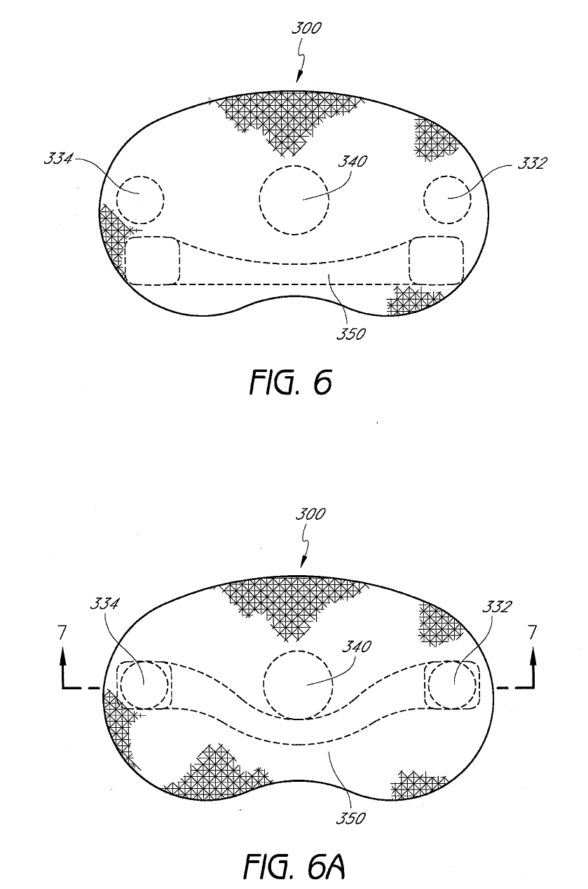

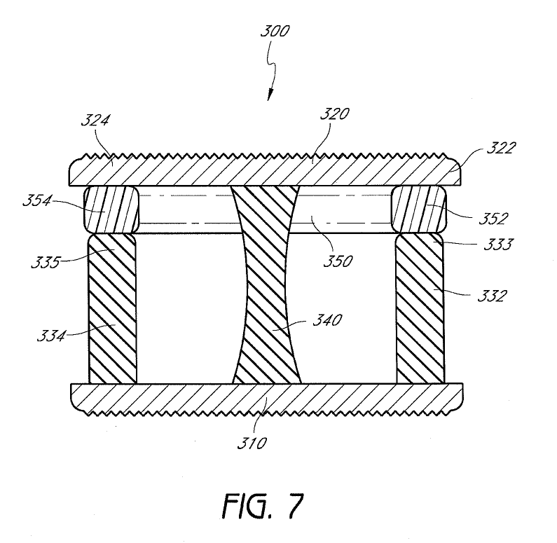

As shown in FIG. 6, an artificial vertebral disc 300 revisable to fusion is provided. Such disc 300 is a modification of the disc 200 described above in connection with FIGS. 4 through 5. In this embodiment, an insert 350 is provided that is designed to be inserted in the space between the distal ends 333, 335 (as shown in FIG. 7) of the stopping members 332, 334 and the upper plate 320. This space can be seen as being open in FIG. 5. As such, the disc 300 provides an artificial vertebral disc which can be fixed between adjacent vertebrae for bone fusion applications. Advantageously, such modification of the disc 200 by insertion of the insert 350, or similar wedge-type pieces, permits the artificial vertebral disc 300 to be used with a variety of bone fusion and bone fixation applications. For example, if the artificial disc shown in FIGS. 4 through 5 provides movement beyond that which is desired, the insert 350 can be inserted to provide a fused or fixed device. In another embodiment, the insert 350 is provided at least partially within the disc 300 and can be moved within the disc 300 so as to also fix the disc 300 for bone fusion applications.

More particularly, the disc 300 comprises plates, an elongate member 340, a first stopping member 332, a second stopping member 334, and an insert 350 configured to be inserted in a space defined by distal surfaces of the stopping members and the inner surface of one of the plates. In one embodiment, the insert 350 comprises a component separate from the artificial disc such that the insert 350 can be delivered to the disc during a spinal operation. In another embodiment, the insert 350 can be integrally associated with the artificial disc 300 such as, for example, connected to a track disposed on an inner surface of the first or second plates. The insert 350 can have a variety of shapes and configurations depending upon a particular application. For example, the insert 350 can be a wedge-type piece in one application or a ramp-type component in another application.

With reference to FIG. 6, a track is disposed on an inner surface of the upper plate. The insert 350 is disposed in a first position along the track such that the insert 350 is not engaged with the stopping members 332, 334. In FIG. 6A, the insert 350 has been moved along the track to a second position, wherein the insert 350 is in an engaged position. In this position, the insert 350 can prevent the upper plate from moving with respect to the lower plate. The insert 350, in one embodiment of the artificial disc 300, generally has a U-shape in order to facilitate movement of the insert 350 with respect to the elongate member 340 centrally disposed within the disc 300.

In one embodiment, the insert 350 is preferably comprised of a resilient material so that the insert 350 can at least partially form around the elongate member 340 when the insert 350 is in an engaged position. Advantageously, movement of the insert 350 within the artificial disc 300 will not be impeded by the elongate member 340. FIG. 6A shows the insert 350 bending at a central portion of the insert 350 at least partially about the elongate member 340 to accommodate moving the insert 350 to an engaged position within the disc 300.

FIG. 7 also shows the insert 350 in an engaged position. The insert 350 has a first end 352 and a second end 354. When the insert is in the second position along the track, for example, the first end 352 of the insert 350 is preferably disposed between a distal end 333 of the first stopping member 332 and a first side 322 of the upper plate 320. Similarly, the second end 354 of the insert 350 is inserted between a distal end 335 of the second stopping member 334 and a second side 324 of the upper plate 320. It can be seen that the insert 350 is generally U-shaped in order to avoid substantial contact with the elongate member 340.

As described in greater detail below with reference to FIGS. 10-17, a method of replacing an intervertebral disc in an interbody space of a spine of a patient can be performed. The method can comprise inserting an access device through an incision in a skin of the patient; expanding said access device from a first configuration to a second configuration, the second configuration having an enlarged cross-sectional area at a distal portion of said access device such that the distal portion extends across at least a portion of the interbody space; and delivering a prosthetic spinal disc implant 300 comprising a first plate 310, an second plate 320, and an elongate member 340 through the access device.

In one embodiment, the prosthetic spinal disc implant 300 further comprises an insert 350 integrally associated with the spinal disc implant 300 and configured to be inserted in a space defined by distal surfaces 333, 335 of the stopping members 332, 334 and one of the plates 310, 320. The disc 300 can include a track disposed on an inner surface of one of the plates 310, 320 wherein the track defines a first position and a second position and the insert 350 is disposed within the track. The method can also include moving the insert 350 along the track from the first position to the second position to prevent one plate from moving relative to the other plate.

In another embodiment, the insert 350 is not integrally associated with the disc 300. In this embodiment, the insert 350 can be delivered through the access device to the disc 300 using suitable surgical instruments. A surgeon can then position the insert in a space defined by distal surfaces 333, 335 of the stopping members 332, 334 and one of the plates to prevent one plate from moving relative to the other plate.

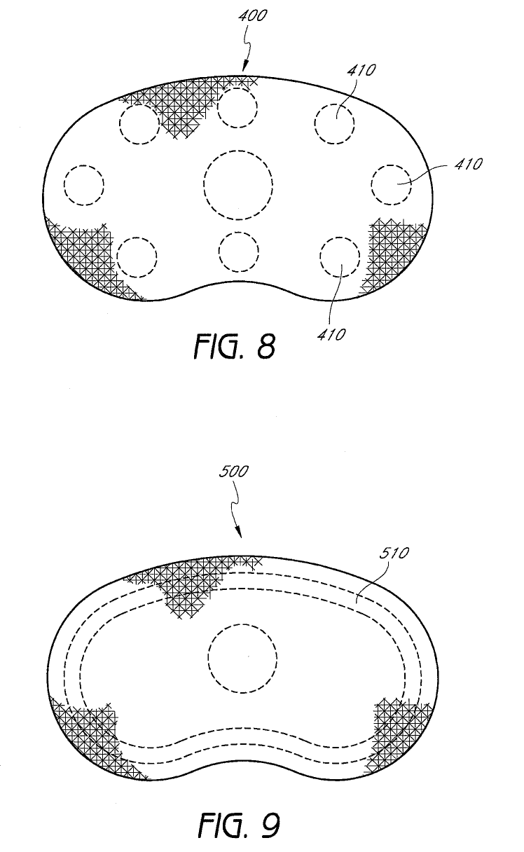

FIGS. 8 and 9 represent open prosthetic discs having stopping members arranged in alternative configurations. In FIG. 8, a series of stopping members 410 is provided such that the stopping members are periodically disposed about the exterior of the disc 400. In FIG. 9, the stopping member comprises a wall 510 extending about the exterior of the artificial disc 500.

Systems, Apparatuses, and Methods for Replacing an Intervertebral Disc

With reference to FIGS. 10 through 17 in general, a wide variety of apparatuses and methods may be used to replace an intervertebral disc in an interbody space of the spine of a patient. For example, an access device can be used to access the disc space. The term “access device” is used in its ordinary sense to mean a device that can provide access and is a broad term and it includes structures having an elongated dimension and defining a passage, e.g., a cannula or a conduit. The access device is configured to be inserted through the skin of the patient to provide access during a surgical procedure to a surgical location within a patient, e.g., a spinal location. The term “surgical location” is used in its ordinary sense (i.e., a location where a surgical procedure is performed) and is a broad term and it includes locations subject to or affected by a surgery. The term “spinal location” is used in its ordinary sense (i.e., a location at or near a spine) and is a broad term and it includes locations adjacent to or associated with a spine that may be sites for surgical spinal procedures. The access device also can retract tissue to provide greater access to the surgical location. The term “retractor” is used in its ordinary sense to mean a device that can displace tissue and is a broad term and it includes structures having an elongated dimension and defining a passage, e.g., a cannula or a conduit, to retract tissue.

Visualization of the surgical site may be achieved in any suitable manner, e.g., by direct visualization, or by use of a viewing element, such as an endoscope, a camera, loupes, a microscope, or any other suitable viewing element, or a combination of the foregoing. The term “viewing element” is used in its ordinary sense to mean a device useful for viewing and is a broad term and it also includes elements that enhance viewing, such as, for example, a light source or lighting element. In one embodiment, the viewing element provides a video signal representing images, such as images of the surgical site, to a monitor. The viewing element may be an endoscope and camera that captures images to be displayed on the monitor whereby the physician is able to view the surgical site as the procedure is being performed.

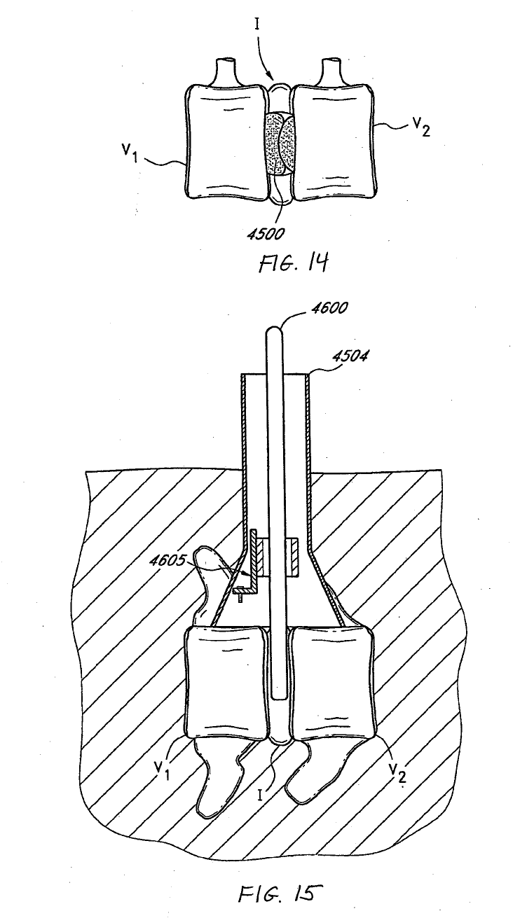

The systems are described herein in connection with minimally invasive postero-lateral spinal surgery. One such procedure, which is not described in comprehensive detail herein, is a two level postero-lateral fixation and fusion of the spine involving the L4, L5, and S1 vertebrae. In the drawings, such as FIGS. 15-17, the vertebrae will generally be denoted by reference letter V. The usefulness of the apparatuses and procedures is neither restricted to the postero-lateral approach nor to the L4, L5, and S1 vertebrae. The apparatuses and procedures may be used in other anatomical approaches and with other vertebra(e) within the cervical, thoracic, and lumbar regions of the spine. The procedures may be directed toward surgery involving one or more vertebral levels. Some embodiments are useful for anterior and/or lateral procedures. A retroperitoneal approach can also be used with some embodiments. In one retroperitoneal approach, an initial transverse incision is made just left of the midline, just above the pubis, about 3 centimeters in length. The incision can be carried down through the subcutaneous tissues to the anterior rectus sheath, which is incised transversely and the rectus is retracted medially. At this level, the posterior sheath, where present, can be incised. With blunt finger dissection, the retroperitoneal space can be entered. The space can be enlarged with blunt dissection or with a retroperitoneal balloon dissector. The peritoneal sack can be retracted, e.g., by one of the access devices described herein.

It is believed that embodiments of the invention are also particularly useful where any body structures must be accessed beneath the skin and muscle tissue of the patient, and/or where it is desirable to provide sufficient space and visibility in order to manipulate surgical instruments and treat the underlying body structures. For example, certain features or instrumentation described herein are particularly useful for minimally invasive procedures, e.g., arthroscopic procedures. As discussed more fully below, one embodiment of an apparatus described herein provides an access device that is expandable, e.g., including an expandable distal portion. In addition to providing greater access to a surgical site than would be provided with a device having a constant cross-section from proximal to distal, the expandable distal portion prevents or substantially prevents the access device, or instruments extended therethrough to the surgical site, from dislodging or popping out of the operative site.

Further embodiments of the present methods and devices for replacement of intervertebral discs, and methods of delivering such devices, can be found in U.S. patent application Ser. No. 10/842,651, filed May 10, 2004, published as U.S. Patent Publication No. 2005/0075644, titled “Methods and Apparatuses for Minimally Invasive Replacement of Intervertebral Discs,” the contents of which are hereby incorporated by reference in its entirety.

A. Systems and Devices for Establishing Access

In one embodiment, the system includes an access device that provides an internal passage for surgical instruments to be inserted through the skin and muscle tissue of a patient to the surgical site. The access device preferably has a wall portion defining a reduced profile, or low-profile, configuration for initial percutaneous insertion into the patient. This wall portion may have any suitable arrangement. In one embodiment, the wall portion has a generally tubular configuration that may be passed over a dilator that has been inserted into the patient to atraumatically enlarge an opening sufficiently large to receive the access device therein.

The wall portion of the access device preferably can be subsequently expanded to an enlarged configuration, by moving against the surrounding muscle tissue to at least partially define an enlarged surgical space in which the surgical procedures will be performed. In a sense, it acts as its own dilator. The access device may also be thought of as a retractor, and may be referred to herein as such. Both the distal and proximal portion may be expanded, as discussed further below. However, the distal portion preferably expands to a greater extent than the proximal portion, because the surgical procedures are to be performed at the surgical site, which is adjacent the distal portion when the access device is inserted into the patient.

While in the reduced profile configuration, the access device preferably defines a first unexpanded configuration. Thereafter, the access device can enlarge the surgical space defined thereby by engaging the tissue surrounding the access device and displacing the tissue outwardly as the access device expands. The access device preferably is sufficiently rigid to displace such tissue during the expansion thereof The access device may be resiliently biased to expand from the reduced profile configuration to the enlarged configuration. In addition, the access device may also be manually expanded by an expander device with or without one or more surgical instruments inserted therein, as will be described below. The surgical site preferably is at least partially defined by the expanded access device itself During expansion, the access device can move from a first overlapping configuration to a second overlapping configuration in some embodiments.

In some embodiments, the proximal and distal portions are separate components that may be coupled together in a suitable fashion. For example, the distal end portion of the access device may be configured for relative movement with respect to the proximal end portion in order to allow the physician to position the distal end portion at a desired location. This relative movement also provides the advantage that the proximal portion of the access device nearest the physician may remain substantially stable during such distal movement. In one embodiment, the distal portion is a separate component that is pivotally or movably coupled to the proximal portion. In another embodiment, the distal portion is flexible or resilient in order to permit such relative movement.

With reference to FIG. 10 in particular, an embodiment of an access device 1000 comprises an elongate body 1020 defining a passage 1040 and having a proximal end 1060 and a distal end 1080. The elongate body 1020 has a proximal portion 1100 and a distal portion 1120. In one embodiment, the proximal portion 1100 has an oblong or generally oval shaped cross section. The term “oblong” is used in its ordinary sense (i.e., having an elongated form) and is a broad term and it includes a structure having a dimension, especially one of two perpendicular dimensions, such as, for example, width or length, that is greater than another and includes shapes such as rectangles, ovals, ellipses, triangles, diamonds, trapezoids, parabolas, and other elongated shapes having straight or curved sides. The term “oval” is used in its ordinary sense (i.e., egg like or elliptical) and is a broad term and includes oblong shapes having curved portions. In other embodiments, the proximal portion 1100 can have a generally circular cross section.

The proximal portion 1100, in one embodiment, comprises an oblong, generally oval shaped cross section over the elongated portion. It will be apparent to those of skill in the art that the cross section can be of any suitable oblong shape (or any other suitable shape). The proximal portion 1100 can be any desired size. The proximal portion 1100 can have a cross-sectional area that varies from one end of the proximal portion to another end. For example, the cross-sectional area of the proximal portion can increase or decrease along the length of the proximal portion 1100. Preferably, the proximal portion 1100 is sized to provide sufficient space for inserting multiple surgical instruments through the elongate body 1020 to the surgical location. The distal portion 1120 preferably is expandable and comprises first and second overlapping skirt members 1140, 1160. The degree of expansion of the distal portion 1120 is determined by an amount of overlap between the first skirt member 1140 and the second skirt member 1160 in one embodiment.

The elongate body 1020 of the access device 1000 has a first location 1180 distal of a second location 1200. The elongate body 1020 preferably is capable of having a configuration when inserted within the patient wherein the cross-sectional area of the passage 1040 at the first location 1180 is greater than the cross-sectional area of the passage 1040 at the second location 1200. The passage 1040 preferably is capable of having an oblong shaped cross section between the second location 1200 and the proximal end 1060. In some embodiments the passage 1040 preferably is capable of having a generally elliptical cross section between the second location 1200 and the proximal end 1060. Additionally, the passage 1040 preferably is capable of having a non-circular cross section between the second location 1200 and the proximal end 1060. Additionally, in some embodiments, the cross section of the passage 1040 can be symmetrical about a first axis and a second axis, the first axis being generally normal to the second axis. In other embodiments, the passage 1040 can have a generally circular cross section.

In another embodiment, an access device comprises an elongate body defining a passage and having a proximal end and a distal end. The elongate body can be a unitary structure and can have a generally uniform cross section from the proximal end to the distal end. In one embodiment, the elongate body preferably has an oblong or generally oval shaped cross section along the entire length of the elongate body. The passage can have a generally elliptical cross section between the proximal end and the distal end. The elongate body preferably has a relatively fixed cross-sectional area along its entire length. In one embodiment, the elongate body is capable of having a configuration when inserted within the patient wherein the cross-sectional area of the passage at a first location is equal to the cross-sectional area of the passage at a second location. The passage preferably is capable of having an oblong shaped cross section between the first and second locations. The cross section of the passage can be of any suitable oblong shape and the elongate body can be any desired size. Preferably, the elongate body is sized to provide sufficient space for inserting multiple surgical instruments sequentially or simultaneously through the elongate body to the surgical location.

In one embodiment, the access device has a uniform, generally oblong shaped cross section and is sized or configured to approach, dock on, or provide access to, anatomical structures. The access device preferably is configured to approach the spine from a posterior position or from a postero-lateral position. A distal portion of the access device can be configured to dock on, or provide access to, posterior portions of the spine for performing spinal procedures, such as, for example, fixation, fusion, or any other suitable procedure. In one embodiment, the distal portion of the access device has a uniform, generally oblong shaped cross section and is configured to dock on, or provide access to, generally posterior spinal structures. Generally posterior spinal structures can include, for example, one or more of the transverse process, the superior articular process, the inferior articular process, and the spinous process. In some embodiments, the access device can have a contoured distal end to facilitate docking on one or more of the posterior spinal structures. Accordingly, in one embodiment, the access device has a uniform, generally oblong shaped cross section with a distal end sized, configured, or contoured to approach, dock on, or provide access to, spinal structures from a posterior or postero-lateral position.

Further details and features pertaining to access devices and systems are described in U.S. Pat. No. 6,800,084, issued Oct. 5, 2004, U.S. Pat. No. 6,652,553, issued Nov. 25, 2003, U.S. application Ser. No. 10/678,744 filed Oct. 2, 2003, published as Publication No. 2005/0075540 on Apr. 7, 2005, which are incorporated by reference in their entireties herein.

B. Apparatuses and Methods for Replacing a Spinal Disc with an Interbody Implant

A type of procedure that can be performed by way of the systems and apparatuses described herein involves replacement of one or more of a patient's spinal discs with an implant, e.g., a prosthetic device, that provides the functions of the spinal disc while preserving or restoring a degree of normal motion after recovery. Such a procedure may be applied to a patient suffering degenerative disc disease or otherwise suffering from disc degeneration. A variety of motion preserving implants that may be applied to replace a damaged or degenerating disc are described below. The access devices and systems described herein enable these devices and methods associated therewith to be practiced minimally invasively.

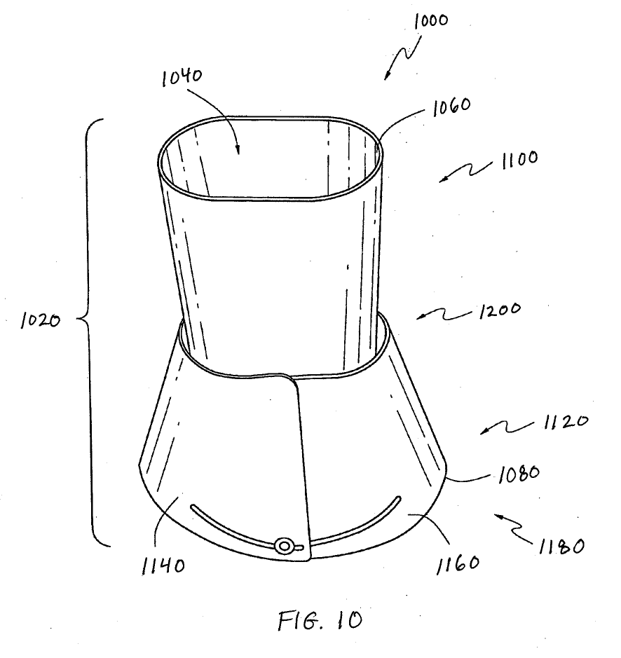

FIGS. 11-17 more particularly illustrate methods whereby an implant 4500 is delivered through an access device 4504 and implanted in an interbody space I defined between a first vertebra V₁ and a second vertebra V₂. The implant 4500 may be any suitable implant, e.g., any of the implants described herein. Some methods of implanting the implant 4500 may be similar to the methods of implanting a fusion implant described in U.S. patent application Ser. No. 10/842,651, filed May 10, 2004, published as U.S. Patent Publication No. 2005/0075644 A1, titled “Methods and Apparatuses for Minimally Invasive Replacement of Intervertebral Discs,” the contents of which are hereby incorporated by reference in its entirety.

In one method, access to the interbody space I is provided by inserting the access device 4504 into the patient. The access device 4504 may be configured in a manner similar to an expandable conduit and may be inserted in a similar manner, e.g., over a dilator. The access device 4504 preferably has an elongate body 4508 that has a proximal end 4512 and a distal end 4516. In one embodiment, the elongate body 4508 comprises a proximal portion 4520 and a distal portion 4524. The proximal portion 4520 may have a generally oblong or oval shape (as shown in FIG. 11A), a generally circular shape (as shown in FIG. 11B), or any other suitable shape. The distal portion 4524 preferably is expandable to the configuration illustrated in FIGS. 11 through 13. At least one passage 4528 extends through the elongate body 4508 between the proximal end 4512 and the distal end 4516.

The elongate body 4508 has a length between the proximal end 4512 and the distal end 4516 that is selected such that when the access device 4504 is applied to a patient during a surgical procedure, the distal end 4516 can be positioned inside the patient adjacent a spinal location, and, when so applied, the proximal end 4512 preferably is located outside the patient at a suitable height. As discussed below, various methods can be performed through the access device 4504 by way of a variety of anatomical approaches, e.g., anterior, lateral, transforaminal, postero-lateral, and posterior approaches. The access device 4504 may be used for any of these approaches and may be particularly configured for any one of or for more than one of these approaches. For example, the access device 4504 may be generally lengthened for certain approaches, e.g., lateral and anterior, compared to other approaches, e.g., posterior and postero-lateral. The access device 4504 may be lengthened by lengthening the proximal portion 4520, the distal portion 4524, or the proximal and distal portions 4520, 4524.

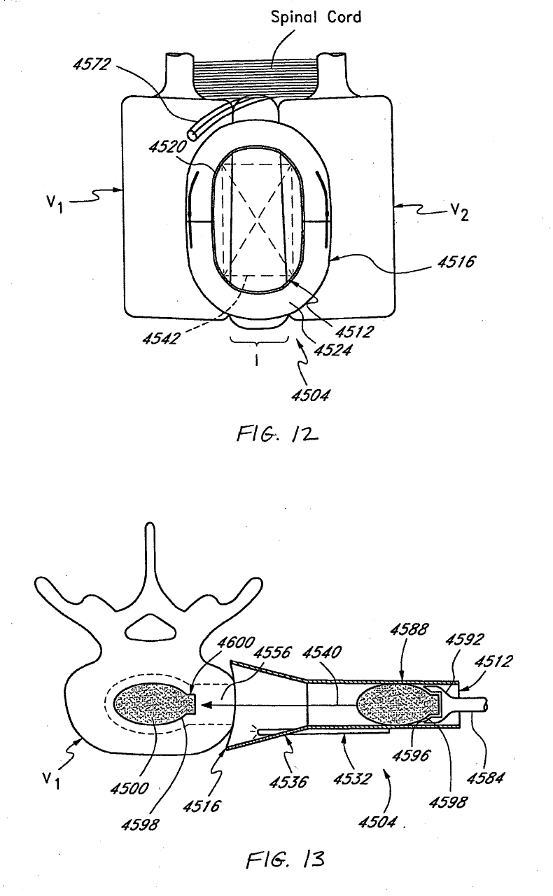

FIG. 13 shows that the access device 4504 is configured to be coupled with a viewing element 4532 in one embodiment. The distal portion 4524 of the access device 4504 has an aperture 4536 into which the viewing element 4532 can be inserted, such that a proximal portion of the viewing element 4532 lies external to the proximal portion 4520 and a distal portion of the viewing element 4532 lies within the distal portion 4524 of the access device 4504. In another embodiment, the viewing element 4532 may extend within the access device 4504 substantially entirely the length of the passage 4528. In other embodiments, the viewing element 4532 may be moved to the surgical location entirely externally to the access device 4504. The viewing element 4532 may be configured to be removed from the access device 4504 during the procedure, as required.

The viewing element 4532 may be any suitable viewing element, such as an endoscope, a camera, loupes, a microscope, a lighting element, or a combination of the foregoing. The viewing element may be an endoscope, such as the endoscope 500, and a camera, which capture images to be displayed on a monitor, as discussed above. Further details of the access device 4504 are set forth in an application entitled “Minimally Invasive Access Device and Method,” filed Oct. 2, 2003, U.S. application Ser. No. 10/678,744, published as Publication No. 2005/0075540 on Apr. 7, 2005, which is hereby incorporated by reference in its entirety.

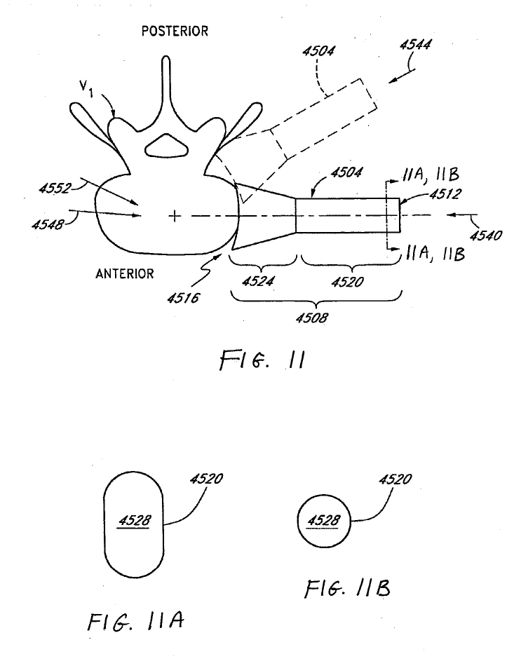

In the illustrated methods, the distal end 4516 of the access device 4504 is inserted laterally, as indicated by an arrow 4540, to a surgical location adjacent to at least one vertebra and preferably adjacent to two vertebrae, e.g., the first vertebra V₁ and the second vertebra V₂, to provide access to at least a portion of the interbody space I. In another method, the access device 4504 is inserted postero-laterally, as indicated by an arrow 4544 and the dashed-line outline of the access device 4504 in FIG. 11, to provide access to at least a portion of the interbody space I. As discussed above, the access device 4504 can have a first configuration for insertion to the surgical location over the interbody space I and a second configuration wherein increased access is provided to the interbody space I. FIGS. 11 and 12 show that the second configuration may provide a cross-sectional area at the distal end 4516 that is larger than that of the first configuration at the distal end 4516. The distal portion 4524 of the access device 4504 may be expanded from the first configuration to the second configuration, as discussed above in connection with the skirt portion 24, using an expander apparatus. When so expanded, the distal portion 4524, at the distal end 4516, defines a surgical space 4542 that includes a portion of the interbody space I, e.g., a portion of the external surface of an annulus A.

As discussed above, in one embodiment, the access device 4504 has a substantially circular cross-sectional shape (as shown in FIG. 11B) in the proximal portion 4520. The access device 4504 may further have a circular cross-section near the proximal end 4512, near the distal end 4516, at the proximal and distal ends 4512, 4516, and from the proximal end 4512 to the distal end 4516. As discussed above, in another embodiment, the access device 4504 has an oblong cross-sectional shape (as shown in FIG. 1A) in the proximal portion 4520. In particular, the access device 4504 may have an oblong cross-section near the proximal end 4512, near the distal end 4516, at the proximal and distal ends 4512, 4516, and from the proximal end 4512 to the distal end 4516.

In some methods of applying the implant 4500, a second access device, such as an expandable conduit or other suitable access device, may be inserted into the patient. For example, a second access device could be inserted through a lateral approach on the opposite side of the spine, as indicated by an arrow 4548, to provide access to at least a portion of an interbody space, e.g., the interbody space I. In another embodiment, a second access device could be inserted through a postero-lateral approach on the opposite side of the spine, as indicated by an arrow 4552, to provide access to at least a portion of an interbody space, e.g., the interbody space I. This second access device may provide access to the interbody space I at about the same time as the first access device 4504 or during a later or earlier portion of a procedure. In one method, the implant 4500 is inserted from both sides of the spine using first and second access devices.

In various applications, one or more implants 4500 may be delivered through one or more access devices, such as the access device 4504, from different directions. For example, a first implant 4500 could be delivered through a first access device from the approach indicated by the arrow 4540, and a second implant 4500 could be delivered through a second access device from the approach indicated by the arrow 4548. In another method, a first implant 4500 could be delivered through a first access device from the approach indicated by the arrow 4540, and a second implant 4500 could be delivered through a second access device from the approach indicated by the arrow 4552. In another method, a first portion of a first implant 4500, e.g., a portion to be coupled with the superior vertebra defining the interbody space I, could be delivered through a first access device from the approach indicated by the arrow 4540, and a second portion of the first implant 4500, e.g., a portion to be coupled with the inferior vertebra defining the interbody space I, could be delivered through a second access device from the approach indicated by the arrow 4548. Thus, any combination of single, multiple implants, or implant sub-components may be delivered through one or more access devices from any combination of one or more approaches, such as the approaches indicated by the arrows 4540, 4544, 4548, 4552, or any other suitable approach.

FIG. 12 shows a lateral view of a portion of a spine of a patient with the access device 4504 delivered thereto. In this figure, the patient's natural disc in the interbody space I has not yet been treated. The access device 4504 is shown in the expanded configuration wherein the perimeter of the distal end 4516 extends outwardly beyond a projection of the perimeter of the proximal end 4512. In one embodiment, the access device 4504 is configured so that when in the expanded configuration, the distal end 4516 does not extend beyond the locations of a nerve root 4572 or the spinal cord. The nerve root 4572 and the spinal cord are located outside the surgical space 4542 defined generally within the perimeter of the distal end 4516 in some embodiments, and therefore are shielded from any implement or implant delivered to the surgical location through the access device 4504. When in position, in addition to providing access to the interbody space I and the disc material therein, the distal portion 4524 may cover the nerve root 4572 and spinal cord and thereby protect the nerve root 4572 and spinal cord. It is understood that the term “cover” as used in this context refers to distal end 4516 of the access device 4504 being located between the surgical space 4542 and the nerve root 4572 or the spinal cord, or in contact with the nerve root 4572 or the spinal cord without applying significant force, e.g., tension or displacement force, to the nerve root 4572 or the spinal cord. The access device 4504 can provide the additional advantage of gently retracting the nerve root 4572 or other delicate anatomical structures where desirable. Gentle retraction of the nerve root 4572 may be desirable in connection with some approaches, e.g., the lateral approach.

As discussed above, in some methods, suitable procedures may be performed to prepare the interbody space I to receive an implant, e.g., the implant 4500. For example, degraded natural disc material may be removed in a suitable manner, e.g., a discectomy may be performed. Also, the surfaces of the vertebrae V₁, V₂ facing the interbody space I may be prepared as needed, e.g., the surfaces may be scraped or scored, and/or holes may be formed in the vertebrae V₁, V₂ to receive one or more features formed on a surface of the implant 4500. FIG. 12 shows a surgical space 4542 wherein an annulotomy and/or end plate removal may be performed through the access device 4504. Such procedures may necessitate the deployment of additional surgical tools through the access device 4504. For example, an annulotomy may be performed using a long handled knife and kerrisons. A discectomy may be completed by using curettes and rongeurs. Removal of osteophytes which may have accumulated between the vertebrae may be performed using osteotomes and chisels. All or only a portion of the disc material within the interbody space I may be removed prior to insertion of the implant 4500. In some methods, the disc material is entirely removed where it will serve no further purpose or will detract from the performance of the implant 4500. Any of the foregoing procedures to prepare the interbody space I may be performed though the access device 4504 inserted as shown or through a second access device inserted through any suitable approach.

In some methods, a distraction means (not shown in FIG. 12) may be provided to further prepare the interbody space I. As indicated by FIG. 13, the distraction means may be used to create a distracted space 4556 in the interbody space I through the same access device used to deliver the implant 4500. The distraction means may take any suitable form, e.g., a paddle distractor, a jacking instrument, etc. Other distraction means known to those of skill in the art could also be used, if configured to be inserted through the access device 4504.

The distracted space 4556 may be formed by manipulating the distraction means to provide a selected separation between the first vertebra V₁ and the second vertebra V₂. The separation and the amount of disc material removed may be selected based on the size of the implant 4500 so as to create sufficient space for the implant 4500 to be received therein. After the distracted space 4556 is formed, the distraction means may be removed to free up the passage 4528 to receive the implant 4500.

In another method, the distraction means is provided through a second access device at about the same time or before the implant 4500 is inserted through the first access device 4504. Any of the approaches described herein or any other suitable approach may be used to deliver the distraction means separately from the implant 4500. In another embodiment, the distraction means is provided through an aperture similar to the aperture 4536 so that the proximal portion of the passage 4528 is unobstructed, and the space therein can be substantially entirely used for the delivery of the implant 4500 during a portion of the method.

FIG. 13 illustrates methods of applying the implant 4500 through the access device 4504. In particular, after the access device 4504 is actuated to the expanded configuration, the implant 4500 is delivered laterally as indicated by the arrow 4540 to a surgical location defined by the distal end 4516 of the access device 4504 at one lateral side of the vertebrae V₁, V₂ and into the interbody space I. In one application, in order to facilitate insertion of the implant 4500, visualization of the surgical site may be achieved in any suitable manner, e.g., by use of a viewing element 4532, as discussed above.

In one procedure, a gripping apparatus 4580, not shown in FIG. 13, is coupled with one or more portions and/or surfaces of the implant 4500 to facilitate insertion of the implant 4500. In one embodiment, the gripping apparatus 4580 has an elongate body 4584 that extends between a proximal end (not shown) and a distal end 4588. The length of the elongate body 4584 is selected such that when the gripping apparatus 4580 is inserted through the access device 4504 to the surgical location, the proximal end extends proximally of the proximal end 4512 of the access device 4504. This arrangement permits the surgeon to manipulate the gripping apparatus 4580 proximally of the access device 4504. The gripping apparatus 4580 has a grip portion 4592 that is configured to engage the implant 4500. In one embodiment, the grip portion 4592 comprises a clamping portion 4596 configured to firmly grasp opposing sides 4598 of the implant. The clamping portion 4596 may further comprise a release mechanism, which may be disposed at the proximal end of the gripping apparatus 4580, to loosen the clamping portion 4596 so that the implant 4500 may be released once delivered to the interbody space I. In another embodiment, the grip portion 4592 comprises a jaw portion with protrusions disposed thereon, such that a portion of the implant 4500 fits within the jaw portion and engages the protrusions. In another embodiment, the grip portion 4592 comprises a malleable material that can conform to the shape of the implant 4500 and thereby engage it. Other means of coupling the gripping apparatus 4580 to the implant 4500 known to those of skill in the art could also be used, if configured to be inserted through the access device 4504.

As shown in FIG. 13, the implant 4500 may be configured to be engaged by the grip portion 4592 of the gripping apparatus 4580. For example, the implant 4500 could include a tab 4600 configured to be engaged by the grip portion 4592 of the gripping apparatus 4580. In one embodiment, the tab 4600 is configured to fit within a jaw portion and engage the protrusions disposed thereon. In another embodiment, the tab 4600 may be configured to fit within a clamping portion 4596 that can be tightened upon it. In another embodiment, the tab 4600 may be configured to mate closely with a corresponding surface in the grip portion 4592 of the gripping apparatus 4580.

In one method of delivering the implant 4500 to the surgical location, the gripping apparatus 4580 is coupled with the implant 4500, as described above. The gripping apparatus 4580 and the implant 4500 are advanced into the proximal end 4516 of the access device 4504, to the surgical space 4542, and further into the interbody space I.

As shown in FIG. 14, in one application, an implant 4500 is delivered into the interbody space I. A first portion of the implant may be delivered to the interbody space I first and thereafter coupled with the lower surface of the superior vertebra V₁ defining the interbody space I. Each of the first and second portions of each of the implants preferably has a generally planar surface. In some embodiments, these surfaces have an element that extends therefrom, which is intended to mate with a corresponding feature, e.g., a hole, formed in the vertebrae V₁, V₂ as discussed above. Next, a second portion of the implant may be delivered to the interbody space I through the same or a different access device, as discussed above, and thereafter coupled with the upper surface of the inferior vertebra V₂ defining the interbody space I.

The implant 4500 may have to be temporarily fixed in place until it becomes secure, e.g., until sufficient bone growth occurs between the adjacent vertebrae V₁, V₂ and one or more surfaces of the implant 4500. In other applications, a structure similar to an endcap could be used to temporarily assist in the securement of the implant 4500 to the adjacent bone structure until the implant 4500 becomes more permanently secure. FIG. 14 shows the spine after the implant 4500 has been inserted between the vertebrae V₁, V₂.

FIGS. 15-17 provide further, detailed methods by which an interbody space may be prepared for the insertion of an implant 4500 delivered through an access device 4504. The methods illustrated are performed via a lateral approach; however, other approaches are also possible, including those enumerated above.

FIG. 15 illustrates the access device 4504 inserted into a patient in a manner such as those discussed above with reference to FIGS. 11-14. Using fluoroscopy in a preferred embodiment to accurately identify the damaged disc, a registration paddle 4600 is inserted through the access device 4504 into the intervertebral disc space. The registration paddle 4600 serves as a place marker to register the location and orientation of the disc that needs to be at least partially replaced with a spinal implant. The registration paddle 4600 preferably has an elongate body that extends between a proximal end and a distal end. The length of the elongate body is selected such that when the registration paddle 4600 is inserted through the access device 4504 to the surgical location, the proximal end extends proximally of the proximal end of the access device 4504, as shown. This arrangement permits the surgeon to manipulate the registration paddle 4600 proximally of the access device 4504. As is well known to those of skill in the art, the registration paddle's distal end corresponds roughly to the shape and size of the interbody space, such that it cannot twist or move easily.

With the registration paddle 4600 accurately positioned and oriented, a guide 4605 is then placed over the registration paddle 4600 and slid down to a location proximal the vertebrae. This guide 4605 may then be attached to a vertebra adjacent the interbody space in a number of ways well-known to those of skill in the art. In one application, the guide 4605 may be inserted using a tool similar to the gripping apparatus 4580 described above. In the illustrated embodiment, the guide 4605 is then screwed into the adjacent vertebral body. As will be appreciated, the guide 4605 will be in a particular location and orientation relative to the intervertebral disc. As a result, subsequent disc preparation and implant insertion procedures can be performed relative to this guide 4605 with greater ease and less reliance on endoscopic apparati. Of course, many other means may be used to affix the guide at various locations and orientations with respect to the interbody space, as is well known to those of skill in the art.

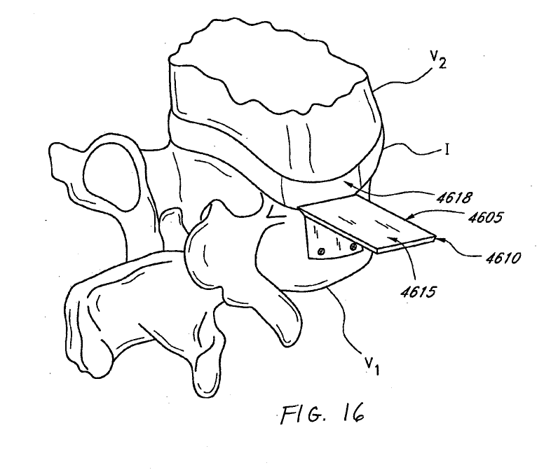

FIG. 16 illustrates in greater detail one embodiment of a guide 4605 in position on a vertebra adjacent an interbody space. The guide 4605 includes a dovetail guide 4610. Other surgical instruments may have corresponding surfaces that engage with this dovetail guide 4610 in order to guide them to the interbody space in a proper orientation. The guide 4605 itself, with its planar surface 4615, also provides orientation and location information to a surgeon. Using this guide 4605, various instruments may be inserted in proper orientation and position relative to the interbody space. In the illustrated embodiment, an annulotomy has been performed directly adjacent the guide 4605, creating an opening 4618 in the spinal disc's annulus.

In other embodiments, other means of locating devices relative to the guide 4605 may be used, including simple grooves and milled paths. In still other embodiments, the guide's surface may not be planar, but may have other geometries that help to guide instruments to the vertebrae. In another embodiment, the guide 4605 may not provide more guidance than its own planar surface running roughly parallel to the intervertebral disc space.

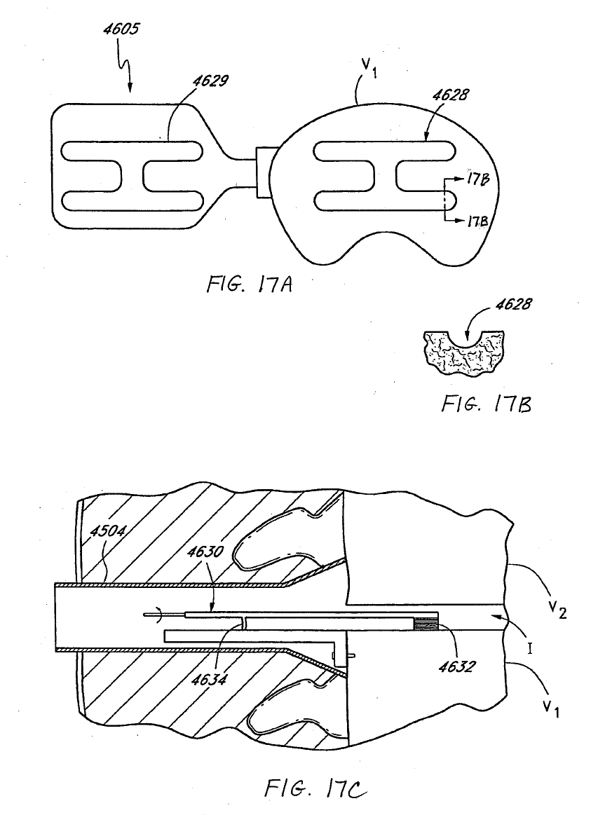

FIGS. 17A, 17B, and 17C show an embodiment of the guide 4605 facilitating the production of milled patterns on the vertebrae in order to facilitate the introduction of a spinal implant. In the illustrated embodiment, the implant to be inserted has an H-formation that faces the vertebral body. If the vertebral body were to have an H-formation 4628 milled from its bone, then the implant would seat better within the disc space and heal more quickly (see FIGS. 17A and 17B).

In the illustrated embodiment, the method of performing this preparatory operation is to have a template 4629 milled in the guide 4605. A mill 4630 is provided that has a cutting edge 4632 at its distal end and a protrusion 4634 near its distal end. The distance chosen between the protrusion 4634 and cutting edge 4632 is chosen to correspond to the distance between the template 4629 in the guide 4605, and a corresponding milled location 4628 in the intervertebral space. Thus, as illustrated in FIG. 17C, the cutting edge 4632 and the protrusion 4634 of the mill 4630 are inserted through the access device 4504. Before cutting, the protrusion 4634 is located within the template 4629 of the guide 4605. The surgeon then follows the template with the mill 4630 in order to make a similar set of milled grooves 4628 within the vertebral body. This process makes the surgical procedure faster and more efficient.

Of course, other uses may also be found for the guide 4605. In one embodiment, not shown, the guide may facilitate the insertion of the implant 4500, by providing the necessary orientation and location information. In another embodiment, the guide 4605 may be used to facilitate the removal or adjustment of an implant that has been previously inserted. In other embodiments, the guide 4605 may be used for a number of other procedures that require knowledge of location and orientation near the spinal column. For example, pedicle screws may be inserted more accurately using the guide 4605, and spinal nucleus replacement may also be facilitated.

Although the forgoing procedures are described in connection with a single level lateral or postero-lateral procedure, other procedures are possible. For example, multiple level disc replacement could be performed with an expandable conduit or other suitable access device. As discussed above, other applications are also possible in which the access device 4504 is not expanded prior to delivery of the implant 4500. In such applications, the access device 4504 remains in the first configuration while the steps described above are performed, or a non-expandable access device may be provided. Also, other approaches could be adopted, e.g., anterior, posterior, transforaminal, or any other suitable approach. In one application, the implant 4500 is inserted at the L5-S1 vertebrae or at the L5-L4 vertebrae anteriorly through the access device 4504. Also, a motion preserving disc replacement procedure could be combined with a fusion procedure in two different interbody spaces, e.g., two adjacent interbody spaces.

Although the methods discussed above are particularly directed to the insertion of an implant 4500, the access device 4504 may also be used advantageously to remove the implant 4500. It may be desirable to remove the implant 4500 if the patient's spine condition changes or if the performance of the implant 4500 is compromised, e.g., through wear or subsidence (reduction in the height of the implant). In one application, the tab 4600 disposed on the implant 4500 may be further configured to facilitate subsequent removal. The gripping apparatus 4580 may also be further configured to facilitate removal as well as insertion. By providing minimally invasive access to the interbody space I, the access device 4504 may be used analogously as described above with reference to the removal of the natural disc material, to remove a previously inserted implant 4500. Upon removal of the implant 4500, various subsequent procedures may be performed in the interbody space I. For example, a new implant 4500 may be inserted through the access device 4504 into the interbody space I. Other procedures that could be performed after removing the previously inserted implant 4500 include the insertion of a fusion device where it is determined that fusion is a more suitable treatment than disc replacement. Such a determination may arise from a change in the condition of the spine, e.g., due to the onset of osteoporosis, that makes disc replacement inappropriate.

Another procedure that may be performed through the access device 4504 involves replacement of two or more joints. Some patients who are suffering from degenerative disc disease also suffer from degenerative facet joint disease. While replacement of both a disc and a facet joint in such a patient is possible during the same operation using other methods, such an operation would be very complicated because it would likely require that the spine be approached both anteriorly and posteriorly. In contrast, in some approaches described hereinabove, the access device 4504 would provide sufficient access to both the interbody space I to facilitate replacement of a disc with the implant 4500 and to one or more facet joints to facilitate replacement of one or more facet joints. For example, the postero-lateral approaches indicated by the arrows 4544, 4552 could provide access to a disc in the interbody space I and an adjacent facet joint. In another method, first and second access devices could be applied in any combination of the lateral and postero-lateral approaches indicated by the arrows 4540, 4548, 4544, and 4552, or other approach, to provide access to a disc in the interbody space I and an adjacent facet joint. In one method three or more joints are replaced, e.g., a disc in the interbody space I and the two corresponding, adjacent facet joints by way of one or more access device applied along any combination of the approaches 4540, 4544, 4548, and 4552, or other approach.

The foregoing methods and apparatuses advantageously provide minimally invasive treatment of disc conditions in a manner that preserves some degree of motion between the vertebrae on either side of the replaced disc. Accordingly, trauma to the patient may be reduced, thereby shortening recovery time. Many of the implants provide a more normal post-recovery range of motion of the spine, which can reduce the need for additional procedures.

It will be understood that the foregoing is only illustrative of the principles of the invention, and that various modifications, alterations, and combinations can be made by those skilled in the art without departing from the scope and spirit of the invention.

1. An artificial vertebral disc for replacing an intervertebral disc of a patient, comprising:
a first plate configured to contact a first vertebra of the spinal column and a second plate configured to contact a second vertebra; and
an elongate member comprising a center post extending between the first plate and the second plate, wherein the elongate member is flexible to allow angulation between the plates.

2. The artificial vertebral disc of claim 1, wherein the first plate comprises a lower plate that contacts a lower vertebra and the second plate comprises an upper plate that contacts an upper vertebra.

3. The artificial vertebral disc of claim 1, wherein the elongate member is compressible.

4. The artificial vertebral disc of claim 1, wherein the elongate member flexes to accommodate a lordotic curvature of the spine.

5. The artificial vertebral disc of claim 1, wherein the elongate member flexes to accommodate a kyphotic curvature of the spine.

6. The artificial vertebral disc of claim 1, further comprising a side support extending along substantially the entire exterior of the artificial disc, wherein the side support, first plate and second plate define a substantially enclosed space, wherein at least a portion of the side support is compressible.

7. The artificial vertebral disc of claim 1, further comprising a stopping member attached to one of the first plate and the second plate and spaced apart from the other of the first plate and second plate, wherein the stopping member limits the angulation between the plates.

8. The artificial vertebral disc of claim 7, wherein the stopping member comprises a first stopping member disposed on a first side of the disc and a second stopping member disposed on a second side of the disc opposite the first side of the disc.

9. The artificial vertebral disc of claim 7, further comprising an insert configured to be inserted in a space defined by a distal surface of the stopping member and the other of the first and second plates.

10. The artificial vertebral disc of claim 9, wherein the insert when inserted in the space defined by the distal surface of the stopping member and the other of the first and second plates prevents one plate from moving relative to the other plate.

11. The artificial vertebral disc of claim 9, wherein the insert comprises a U-shaped member.

12. The artificial vertebral disc of claim 1, further comprising means for securing the first and second plates to the first and second vertebra.

13. An artificial vertebral disc for replacing an intervertebral disc of a patient, comprising:
a first plate configured to contact a first vertebra of the spinal column and a second plate configured to contact a second vertebra;
an elongate member connecting the first plate and the second plate, wherein the elongate member allows the second plate to move relative to the first plate; and
a side support extending along substantially the entire exterior of the artificial disc, wherein the side support, first plate and second plate define a substantially enclosed space, wherein at least a portion of the side support is compressible.

14. The artificial vertebral disc of claim 13, wherein the first plate comprises a lower plate that contacts a lower vertebra and the second plate comprises an upper plate that contacts an upper vertebra.

15. The artificial vertebral disc of claim 13, wherein the elongate member comprises a center post.

16. The artificial vertebral disc of claim 13, wherein the elongate member is flexible.

17. The artificial vertebral disc of claim 13, wherein the elongate member is compressible.

18. The artificial vertebral disc of claim 13, wherein the elongate member comprises a nickel-titanium alloy.

19. The artificial vertebral disc of claim 13, wherein the first plate, second plate, elongate member, and side support define a cavity.

20. The artificial vertebral disc of claim 19, wherein rubberized material substantially fills the cavity of the artificial disc to replicate the natural characteristics of an intervertebral disc.

21. The artificial vertebral disc of claim 13, further comprising a sheath covering an exterior surface of the side support and extending around the exterior of the disc.

22. The artificial vertebral disc of claim 13, wherein the elongate member flexes to accommodate a lordotic curvature of the spine.

23. The artificial vertebral disc of claim 13, wherein the elongate member flexes to accommodate a kyphotic curvature of the spine.

24. The artificial vertebral disc of claim 13, further comprising means for securing the first and second plates to the first and second vertebra.

25. The artificial vertebral disc of claim 24, wherein the means for securing the first and second plates to the first and second vertebra comprises rough surfaces adapted to engage vertebrae.

26. The artificial vertebral disc of claim 24, wherein the means for securing the first and second plates to the first and second vertebra comprises spikes.

27. An artificial vertebral disc for replacing an intervertebral disc of a patient, comprising:
a first plate configured to contact a first vertebra of the spinal column and a second plate configured to contact a second vertebra;
an elongate member connecting the first plate and the second plate, wherein the elongate member allows one plate to move relative to the other plate; and
a stopping member attached to one of the first plate and the second plate and spaced apart from the other of the first plate and second plate, wherein the stopping member limits the angulation of the other of the first plate and the second plate.

28-50. (canceled)

51. A method of replacing an intervertebral disc in an interbody space of a spine of a patient, comprising:
inserting an artificial vertebral disc in the interbody space between a first vertebra and a second vertebra, wherein the artificial disc comprises a first plate that contacts the first vertebra and a second plate that contacts the second vertebra and an elongate member comprising a center post extending between the first plate and the second plate, wherein the elongate member is flexible to allow angulation between the plates.
